(19) United States
(12) Patent Application Publication
(10) Pub. No.: US 2022/0000952 A1
(43) Pub. Date: Jan. 6, 2022

(54) METHOD PREPARING ANTRODIA CINNAMOMEA EXTRACT AND ANTRODIA CINNAMOMEA COMPOSITION, AND PHARMACEUTICAL COMPOSITION

(71) Applicant: YONG TENG BIOTECHNOLOGY CO. LTD., New Taipei City (TW)

(72) Inventors: Chen-Han CHUNG, New Taipei City (TW); Chia-Ying CHUNG, New Taipei City (TW)

(21) Appl. No.: 17/292,610

(22) PCT Filed: Aug. 26, 2019
(86) PCT No.: PCT/CN2019/102475

§ 371 Date: May 10, 2021

(30) Foreign Application Priority Data
Nov. 14, 2018 (CN) 201811353402.4

(51) Int. Cl. A61K 36/07; A61P 35/00

(52) U.S. Cl. CPC A61K 36/07; A61P 35/00; A61K 2236/53; A61K 2236/51; A61K 2236/333

(57) ABSTRACT

A method for preparing an Antrodia cinnamomea extract, comprising: using an ethanol solution to extract culture dish-type Antrodia cinnamomea sporocarp powder, concentrating a filtrate under reduced pressure to obtain a crude extract; adsorbing the crude extract by using a macroporous resin and then placing same in a rectangular container; sequentially eluting by using a mixed solution, concentrating an eluate under reduced pressure, and drying to obtain a first sub-extract of an Antrodia cinnamomea extract; next, eluting by using an ethanol solution, concentrating an eluate under reduced pressure, and drying to obtain a second sub-extract of the Antrodia cinnamomea extract; next, eluting by using an ethyl acetate solution, concentrating an eluate under reduced pressure, and drying to obtain a third sub-extract of the Antrodia cinnamomea extract. The obtained sub-extracts of an Antrodia cinnamomea extract may be used for treating cancer and reducing the side effects of chemotherapy.

### TECHNICAL FIELD

The present invention relates to the technical field of edible medicinal fungi, in particular to a method preparing Antrodia cinnamomea extract and Antrodia cinnamomea composition for treating cancer and alleviating side effects of chemotherapy, and a pharmaceutical composition.

### BACKGROUND OF THE INVENTION

Antrodia cinnamomea belongs to a non-bacterial mesh, a porous bacterial material, a thin-pore strain, an Antrodia cinnamomea seed and a perennial mushroom fungus. It is an endemic fungus in Taiwan. The present research results show that the Antrodia cinnamomea has various functions of resisting tumors, enhancing immunity, resisting viruses, resisting inflammation, resisting oxidation and protecting liver. Among them, the researches and patents related to anti-tumor are mostly focused on the direct application of basswood-cultivated Antrodia cinnamomea sporocarp or mycelial powder of Antrodia cinnamomea or extracts of different solvents or a single compound of Antrodia to the treatment of cancer cells.

Due to the fact that the number of wild Antrodia cinnamomea is scarce and is not easy to grow, and there is a problem of environmental pollution of the growth environment, the Antrodia cinnamomea is cultured in a plurality of artificial cultivation methods in recent years. At present, the Antrodia cinnamomea cultivation method is mainly produced by a liquid culture method, a solid state culture method and a basswood culture method. The type and composition of Antrodia cinnamomea can vary greatly due to different cultivation methods, and the Antrodia cinnamomea mycelium is obtained by culturing in liquid fermentation and solid state fermentation described in the literature, and the Antrodia cinnamomea sporocarp is obtained by culturing the basswood. The components of the Antrodia cinnamomea mycelium are mainly composed of a polysaccharide, and the components of the Antrodia cinnamomea sporocarp are mainly triterpenes. The price of the Antrodia cinnamomea sporocarp by culturing the basswood is very expensive due to the fact that basswood of the Antrodia cinnamomea is not easy to obtain.

The Cancer is a disease that is prevalent throughout the world with high lethal rates, and the treatment of the cancer includes surgery, chemotherapy, radiation therapy, targeted treatment and combinations of these treatment methods. Although the chemotherapy can kill cancer cells successfully, it will kill normal cells non-selectively, and there will typically be a serious side effect on the patient; chemotherapy drugs cause a low number of leukocytes in the patient to be one of the common side effects, and the number of leukocytes is often too low to affect the treatment during the acceptance of the chemical treatment for cancer patients. Therefore, traditional Chinese herbal medicine extracts with anticancer activity are found from traditional Chinese herbal medicines to replace western medicines or combined with western medicines to treat cancer as a new direction worthy of research.

### SUMMARY OF THE INVENTION

In order to solve the problem of side effects caused by the treatment of cancer and chemical treatment on patients in the process of cancer treatment, the invention provides a method for preparing an Antrodia cinnamomea extract, comprising the following steps: using an ethanol solution to extract culture dish-type Antrodia cinnamomea sporocarp powder, concentrating a filtrate under reduced pressure to obtain a crude extract; adsorbing the crude extract by using a macroporous resin and then placing same in a rectangular container; sequentially eluting by using a mixed solution, concentrating an eluate under reduced pressure, and drying to obtain a first sub-extract of an Antrodia cinnamomea extract; next, eluting by using an ethanol solution, concentrating an eluate under reduced pressure, and drying to obtain a second sub-extract of the Antrodia cinnamomea extract; next, eluting by using an ethyl acetate solution, concentrating an eluate under reduced pressure, and drying to obtain a third sub-extract of the Antrodia cinnamomea extract.

The mixed solution is composed of a secondary water and an ethanol solution; the volume ratio of the secondary water to the ethanol solution is (40-60): (40-60).

The volume fraction of the ethanol solution is 95%.

The invention provides a method for preparing an Antrodia cinnamomea composition, comprising the following steps: extracting Antrodia cinnamomea mycelium powder by using a secondary water, concentrating a filtrate under reduced pressure to obtain a concentrated solution; adding the concentrated solution into an ethanol solution, filtering to obtain a precipitate; drying the precipitate to obtain an Antrodia cinnamomea mycelium extract; and combining the Antrodia cinnamomea mycelium extract with a second sub-extract of the Antrodia cinnamomea extract prepared by the method of claim 1 to obtain an Antrodia cinnamomea composition.

The weight ratio of the Antrodia cinnamomea mycelium extract to the second sub-extract of the Antrodia cinnamomea extract is (35-65): (35-65).

The volume ratio of the concentrated solution to the ethanol solution is 1: 3; the volume fraction of the ethanol solution is 60%-80%.

The invention provides a pharmaceutical composition comprising a crude extract prepared by the above method at a therapeutic effective dose and a pharmaceutically acceptable carrier as appropriate, and is used for preparing medical drugs for preventing or treating colorectal cancer, liver cancer and skin cancer; the medical drug has poison killing effect on colorectal cancer, liver cancer and skin cancer cells.

The invention provides a pharmaceutical composition comprising a second sub-extract or a third sub-extract of .Antrodia cinnamomea extract prepared by the above method at a therapeutic effective dose, and a pharmaceutically acceptable carrier as appropriate, and is used for preparing medical drugs for preventing or treating colorectal cancer, liver cancer, gastric cancer, lung cancer, breast cancer and skin cancer; the medical drug has poison killing effect on colorectal cancer, liver cancer, gastric cancer, lung cancer, breast cancer and skin cancer cells.

The invention also provides a pharmaceutical composition comprising an Antrodia cinnamomea composition prepared by the above method at a therapeutic effective dose, and a pharmaceutically acceptable carrier as appropriate, and is used for preparing drugs for improving the side effects of the chemotherapeutic drug cisplatin and the platinum-containing series of drugs; the drug is able to improve the number of leukocytes and the phenomenon of bone marrow inhibition caused by cisplatin series drugs of chemotherapy drugs.

The sub-extract of Antrodia cinnamomea extract or Antrodia cinnamomea composition prepared by using the method provided by the invention can be used for treating cancer and relieving side effects of chemotherapy, including the treatment of liver cancer, lung cancer, colorectal cancer, gastric cancer, breast cancer, skin cancer and so on, and has a remarkable curative effect on improving the number of leukocytes and bone marrow inhibition caused by chemotherapy drugs.

### BRIEF DESCRIPTION OF THE DRAWINGS

FIG. 1 is a flow chart of a method for preparing Antrodia cinnamomea extract provided in the preferred embodiment of the present invention;

FIG. 2 is a flow chart of a method for preparing Antrodia cinnamomea mycelium extract provided in the preferred embodiment of the present invention;

FIG. 3 is a graph of the efficacy of the sub-extract (F2) of Antrodia cinnamomea extract for tumor efficacy inhibition of human colon cancer cells HCT116 in animal model in the preferred embodiment of the present invention;

FIG. 4 is the effect of the sub-extract (F2) of the Antrodia cinnamomea extract on the body weight of the human colon cancer cell HCT116 in animal model in the preferred embodiment of the present invention;

FIG. 5 is the effect of the Antrodia cinnamomea composition on the low number of white blood cells caused by cisplatin in C57BL/6 immune normal mice in the preferred embodiment of the present invention;

FIG. 6 is the effect of the Antrodia cinnamomea composition on leukocyte precursor cells of C57BL/6 immune normal mice treated with cisplatin in the preferred embodiment of the present invention;

FIG. 7 is the effect of the Antrodia cinnamomea composition on the red blood cell precursor cells of C57BL/6 immune normal mice treated with cisplatin in the preferred embodiment of the present invention.

### DETAILED DESCRIPTION OF THE PREFERRED EMBODIMENTS

The technical scheme of the present invention is further described below through the accompanying drawings and the preferred embodiment.

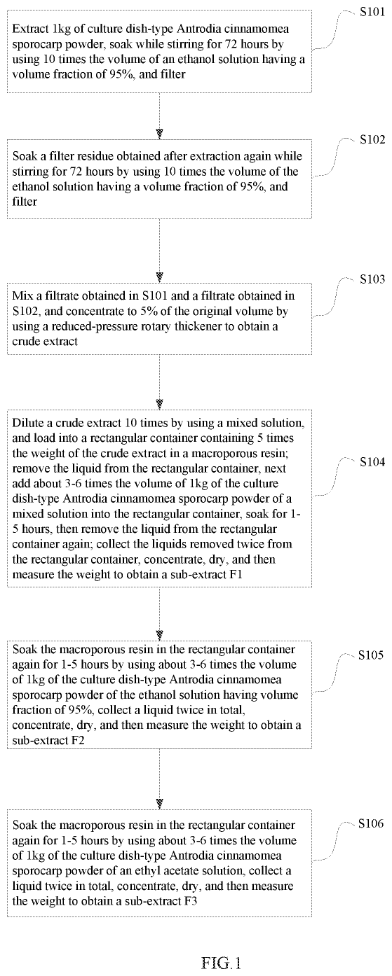

Referring to FIG. 1, the Antrodia cinnamomea extract provided in the preferred embodiment of the present invention is obtained by extracting culture dish-type Antrodia cinnamomea . sporocarp through an ethanol solution, adsorbing by using a macroporous resin and then placing in a rectangular container for partial purification to obtain an extract. The method for preparing an Antrodia cinnamomea extract comprises the following steps:

Step S101: taking 1 kg of dish-cultured Antrodia cinnamomea sporocarp powder, soaking and stifling with a 10-fold volume fraction of ethanol solution with a volume fraction of 95% for 72 hours, and filtering;

Step S102: the filter residue obtained after the step S101 is extracted is soaked and stirred with a 10-fold volume fraction of ethanol solution with a volume fraction of 95% for 72 hours, and filtering;

Step S103: mixing the filtrate obtained in step S101 and the filtrate obtained in step S102, and concentrating the filtrate to 5% of the original volume by using a reduced-pressure rotary concentrator to obtain a Crude extract;

Step S104: diluting the crude extract obtained in step S103 with a mixed solution for 10 times, pouring into a rectangular container containing a macroporous resin (e.g., DIAION® HP20) containing about 5 times the weight of the crude extract, and sufficiently stirring to allow the macroporous resin to adsorb the crude extract (the adsorption time is 10 to 12 hours); pouring out the liquid in the rectangular container, adding a mixed solution of approximately 3-6 times the volume of the 1 kg dish-cultured Antrodia. cinnamomea sporocarp powder into a rectangular container, soaking for 1-5 hours, and pouring out the solution in the rectangular container again; collecting the liquid poured out from the rectangular containers twice, and concentrating by using a reduced-pressure rotary concentrator and (frying, and weighing the weight to obtain the sub-extract F1 of the Antrodia cinnamomea extract;

wherein the mixed solution is composed of a secondary water and an ethanol solution with a volume fraction of 95%, wherein the volume ratio of the secondary water to the ethanol solution with a volume fraction of 95% is (40-60): (40-60);

Step S105: soaking the macroporous resin in the rectangular container with an ethanol solution of approximately 3-6 times the volume of the 1 kg dish-cultured Antrodia. cinnamomea sporocarp powder with a volume fraction of 95% for 1-5 hours, collecting the liquid twice, and concentrating by using a reduced-pressure rotary concentrator and drying, and weighing the weight to obtain the sub-extract F2 of the Antrodia cinnamomea extract;

Step S106: soaking the macroporous resin in the rectangular container with an ethyl acetate solution of approximately 3-6 times the volume of the 1 kg dish-cultured Antrodia. cinnamomea sporocarp powder for 1-5 hours, collecting the liquid twice, and concentrating by using a reduced-pressure rotary concentrator and drying, and weighing the weight to obtain the sub-extract F3 of the Antrodia cinnamomea extract.

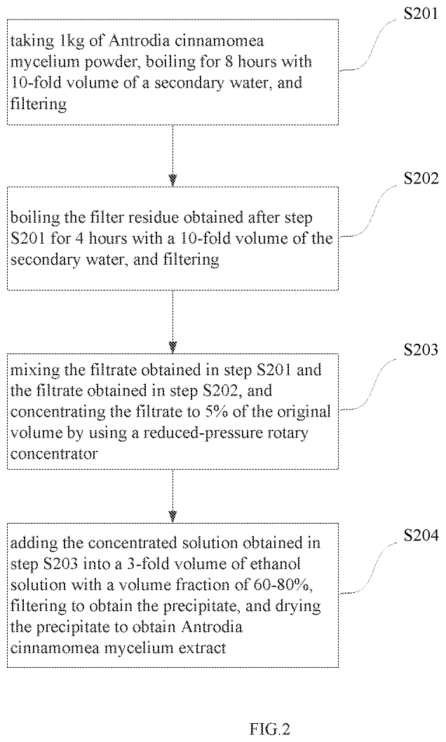

Referring to FIG. 2, the Antrodia cinnamomea mycelium extract in the preferred embodiment of the present invention is obtained by using a secondary water to extract Antrodia cinnamomea mycelium powder, adding the filtered concentrated solution into an ethanol solution, and drying the filtered precipitate to obtain Antrodia cinnamomea mycelium extract. The method for preparing an Antrodia cinnamomea mycelium extract comprises the following steps:

Step S201: taking 1 kg of Antrodia cinnamomea. mycelium powder, boiling for 8 hours with 10-fold volume of a secondary water, and filtering;

Step S202: boiling the filter residue obtained after step S201 for 4 hours with a 10-fold volume of the secondary water, and filtering;

Step S203: mixing the filtrate obtained in step S201 and the filtrate obtained in step S202, and concentrating the filtrate to 5% of the original volume by using a reduced-pressure rotary concentrator;

Step S204: adding the concentrated solution obtained in step S203 into a 3-fold volume of ethanol solution with a volume fraction of 60-80%, filtering to obtain the precipitate, and drying the precipitate to obtain Antrodia cinnamomea mycelium extract.

The Antrodia cinnamomea mycelium extract and the sub-extract F2 of the Antrodia cinnamomea extract in the preferred embodiment of the present invention are combined according to the volume ratio (35-65): (35-65) to obtain the Antrodia cinnamomea composition provided in the preferred embodiment of the present invention.

In order to further illustrate the preparation process of the Antrodia cinnamomea composition provided in the preferred embodiment of the present invention, specific application embodiments are given below.

### Embodiment 1: Preparation of Antrodia Cinnamomea Extract

1 kg of dish-cultured Antrodia cinnamomea sporocarp powder was taken, and was soaked and stirred for 72 hours with a 10-fold volume fraction of an ethanol solution with a volume fraction of 9:5%, and the first filtrate was collected by suction filtration. The filtered filter residue was again soaked and stirred for 72 hours with a 10-fold volume fraction of an ethanol solution with a volume fraction of 95%, and the second filtrate was collected by suction filtration. The first filtrate and the secondary filtrate was combined and concentrated under reduced pressure to obtain the Antrodia cinnamomea ethanol crude extract. The crude extract was diluted by 10 times with a secondary water and an ethanol solution with a volume fraction of 95% (the volume ratio of the secondary water to the ethanol solution with a volume fraction of 95% was 40:60) , and poured into a rectangular container containing a macroporous resin (e.g., DIAION® HP20) containing about 5 times the weight of the crude extract, and sufficiently stirred to allow the macroporous resin to adsorb the crude extract (the adsorption time is 10 to 12 hours). The liquid in the rectangular container was poured out, and a mixed solution (the mixed solution was composed of a secondary water and an ethanol solution with a volume fractions of 95%, and the volume ratio of the secondary water to the ethanol solution with a volume fraction of 95% was 40: 60) of approximately 3-6 times the volume of the 1 kg dish-cultured Antrodia cinnamomea sporocarp powder was added into the rectangular container. After 1-5 hours of soaking, the solution in the rectangular container was poured out, the filtrate was collected twice, concentrated and dried, and weighed to obtain the sub-extract F1 of Antrodia cinnamomea extract. The macroporous resin in the rectangular container was soaked with an ethanol solution of approximately 3-6 times the volume of the 1kg dish-cultured Antrodia cinnamomea sporocarp powder with a volume fraction of 95% again for 1-5 hours, the liquid was collected twice, concentrated and dried, and weighed to obtain the sub-extract F2 of Antrodia. cinnamomea extract. The macroporous resin in the rectangular container was soaked with an ethyl acetate solution of approximately 3-6 times the volume of the 1kg dish-cultured Antrodia cinnamomea sporocarp powder again for 1-5 hours, the liquid was collected twice, concentrated and dried, and weighed to obtain the sub-extract F3 of Antrodia cinnamomea extract.

It should be noted that the volume ratio of the secondary water to the ethanol solution with a volume fraction of 95% in the mixed solution may also be 45: 55, 50: 50, 55: 45, or 60: 40 and so on in practical applications. For the secondary water with different volume ratios and an ethanol solutions with a volume fraction of 95% in the mixed solution, the present embodiments are not described in detail separately.

### Embodiment 2: Preparation of Antrodia Cinnamomea Mycelium Extract

1 kg of Antrodia cinnamomea mycelium powder was taken, and was boiled for 8 hours with a 10-fold volume of a. secondary water, and filtered. The filtered filter residue was boiled again with a 10-fold volume of a secondary water for 4 hours, and filtered. The twice obtained filtrate was mixed and concentrated to 5% of the original volume by using a reduced-pressure rotary concentrator. The obtained concentrated solution was added with a 3-fold volume of ethanol solution with a volume fraction of 70%, the precipitate was taken after filtration, and the precipitate was dried to obtain the Antrodia cinnamomea mycelium extract.

It should be noted that the volume fraction of the concentrated solution to 3 times the volume of ethanol solution may also be 60%, 65%, 75%, or 80% and so on in practical applications. For an ethanol solution with different volume fractions added to the concentrated solution, the present embodiments are not described in detail separately.

### Embodiment 3: Preparation of Antrodia Cinnamomea Composition

The Antrodia cinnamomea mycelium extract obtained in embodiment 2 and the sub-extract F2 of the Antrodia cinnamomea extract obtained in embodiment 1 were combined according to a volume ratio of 35: 65 to obtain an Antrodia. cinnamomea composition (AC).

It should be noted that the volume ratio of the Antrodia cinnamomea mycelium extract to the sub-extract F2 of the Antrodia cinnamomea extract can also be 40: 60, 45: 55, 50: 50, 55: 45, 60: 40 or 65: 35 and so on in practical applications. For different volume ratios of the Antrodia cinnamomea mycelium extract and the sub-extract F2 of the Antrodia cinnamomea extract, the present embodiment is not described in detail separately.

The sub-extract of Antrodia. cinnamomea extract or Antrodia cinnamomea composition prepared by using the method provided in the preferred embodiment of the present invention can be used for treating cancer and relieving side effects of chemotherapy, including the treatment of liver cancer, lung cancer, colorectal cancer, gastric cancer, breast cancer, skin cancer and so on, and has a remarkable curative effect on improving the number of leukocytes and bone narrow inhibition caused by chemotherapy drugs. The following is illustrated by some test results.

1. Antrodia Cinnamomea Extract for Cytotoxicity Assay of Different Cancer Cells

1) Cell culture

HCT116 human colorectal cancer cells, Huh7 human liver cancer cells, MKN45 human gastric cancer cells, A549 human lung cancer cells, MDA-MB-231 human breast cancer cells and A375 human skin cancer cells were selected. HCT 116, Huh7, A549, MDA-MB-231 and A375 cells were cultured in DMEM medium containing 10%13S at 37° C. in a humidified incubator containing 5% CO₂, and MKN45 cells were cultured in RPMI medium containing 10% FBS at 37° C. in a humidified incubator containing 5% CO₂. Vaccinating 1.2×10⁶ cells at T75 flask, the culture dish could be full of cells in about three to seven days according to the growth rate of the cells. When subculturing, removing the culture medium, washing the cells with PBS, adding trypsin-EDTA to peel off the cells, neutralizing in the new culture medium, counting the cells, vaccinating the appropriate number of cells in the culture dish according to the experimental requirements, and remaining 1.2×10⁶ cells to vaccinate into the new T75 flask for continued culture.

2) Configuration of the Test Drug

The test substances including Antrodia cinnamomea extract were labeled as the samples of Crude, F1 , F2 and F3 by using 100% DMSO, and were configured at a concentration of 50 mg/mL. The test drugs were diluted to the following eight concentrations: 2000,1500,1000,500,250,50,25 and 12.5 μg/mL in DMEM or RPMI medium. The above concentrations were ten times of the final concentration

3) Cell Survival Assay (MTS Assay)

6×10³ cells were implanted in each well in a 96-well plate, cultured at 37° C. for 4 hours, and the test drugs was added into the wells. The experiment was repeated three times for each concentration. After incubation at 37° C. for 48 hours, the old culture solution was removed, the culture solution containing MTS was added, the reaction was allowed to react at 37° C. for 1 hour, the absorbance value of the wavelength 490 nm was read by ELISA Reader, and the IC₅₀ value was calculated using GraphPad Prism 5 software. The results were shown in Table 1 below.

TABLE 1

| | Sample | | | |
|---|---|---|---|---|
| In vitro potency | Crude | F1 | F2 | F3 |
| Cell line | Antiproliferative activity (IC₅₀, g/mL) | | | |
| Huh7 (liver cancer) | 141.3 | >200 | 16.1 | 36.7 |
| A549 (lung cancer) | >200 | >200 | 68.4 | 193.0 |
| HCT116 (colon cancer) | 120.2 | >200 | 75.9 | 128.8 |
| MKN-45 (gastric cancer) | >200 | >200 | 28.2 | 92.5 |
| MDA-MB231(breast cancer) | >200 | >200 | 54.9 | >200 |
| A375 (skin cancer) | 162.7 | >200 | 10.1 | 115.6 |

The results of Table 1 show the Crude extract of Antrodia cinnamomea extract (Crude) has no poison killing effect on A549 (lung cancer), MKN-45 (gastric cancer) and MDA-MB231 (breast cancer), and has weak poison killing effect on Huh7 (liver cancer), HCT116 (colon cancer) and A375 (skin cancer). The sub-extract (F1) of Antrodia cinnamomea extract has no poison killing effect on the six cancer cells tested. The sib-extract (F2) of Antrodia cinnamomea extract has strong poison killing effect on six cancer cells tested, especially on A375 (skin cancer), Huh7 (liver cancer) and MIKN-45 (gastric cancer) poisoning efficacy. The sub-extract (F3) of Antrodia cinnamomea extract has no poison killing effect on MDA-MB231 (breast cancer) in six cancer tested, has strong poison killing effect on Huh7 (liver cancer), and has weak poison killing effect on the other four cancer cells.

2. Animal-model efficacy test of Antrodia cinnamomea extract on human colon cancer cell HCT116

1) Feeding of Experimental Animals

The animals used in this study were BALB/cnu/nu nude mice which were commercially available from Taiwan Experimental Animal Centers and six weeks old. The feeding temperature was controlled to be 25±2° C., the illumination time was 08: 00-20: 00, sufficient feed and drinking water were provided daily, and the padding was replaced twice a week.

2) Tumor-Induced Animal Patterns

The human colon cancer cell strain (HCT116) was suspended in 80 LPBS at a cell mass of 3×10⁶ and implanted in subcutaneous sites on the right side of the back of the nude mice in an injection manner to induce tumor generation until the tumor size reached 100-200mm³ as the benchmark, and starting dosing.

3) Test Substance and Route of Administration

The sub-extract F2 was administered in a tube-feed manner on the 1st to 15th day. The animal groups were the control group, 100, 200 and 400 mg/kg F2 respectively, with 6 mice in each group and 24 mice in 4 groups.

4) Evaluation of Inhibition of Tumor Growth

The mice were sacrificed in a CO₂ manner, the tumor sites were weighed after surgery, and the tumor type and weight change after administration were compared. If the experimental animal died before the sacrificial day, the death time was recorded as a basis for comparison of life extension after drug administration.

5) Statistical Analysis Methods

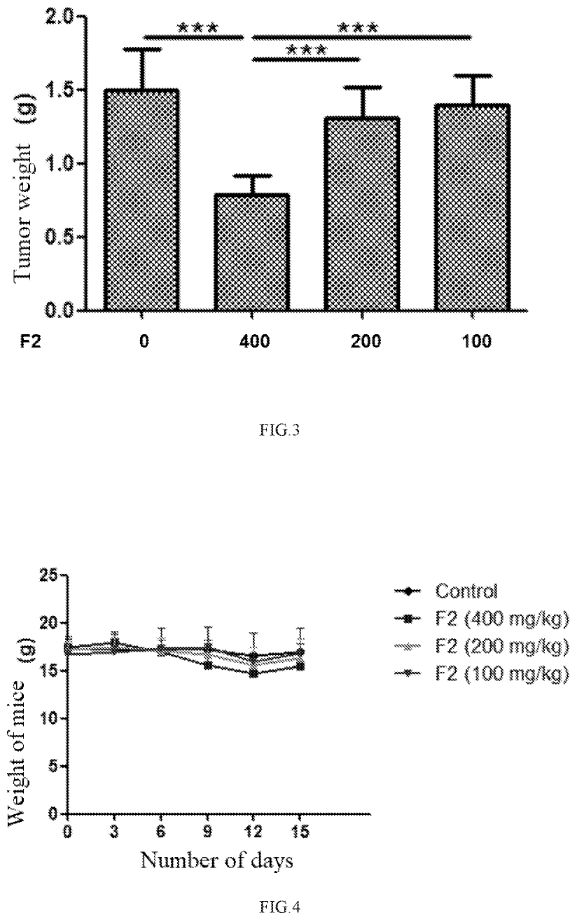

One-way analysis of variance (One-Way ANOVA) was used for data analysis. When the analysis result reached a significant level, Fisher LSD was used for subsequent comparison. The results of animal experiments are shown in FIG. 3 and FIG. 4.

The results in FIG. 3 show that the sub-extract (F2) of Antrodia cinnamomea extract had a dose effect on tumor inhibition when different doses of the sub-extract (F2) were orally administered to immune deficient mice implanted in human colon cancer cells HCT116. There were obvious statistical differences in the dose of 400 mg/kg group. This result shows that 400 mg/kg was a therapeutically effective dose for the treatment of immune deficient mice implanted with human colon cancer cells HCT116. The results in FIG. 4 show that three administration doses of the sub-extract (F2) of the Antrodia cinnamomea extract did not cause the weight loss of the immune deficient mice implanted with the human colon cancer cell HCT116. This result shows that the sub-extract (F2) of the Antrodia cinnamomea extract did not cause the side effects of body weight loss in inhibiting the tumor.

3. Efficacy Test of Antrodia Cinnamomea Composition for Improving the Bone Marrow Inhibition of Chemotherapeutic Drug Cisplatin in C57BL/6 Immune Normal Mice

1) Feeding of Experimental Animals

The animals used in this study were C57B116 mice which were commercially available from Taiwan Experimental Animal Centers and six weeks old. The feeding temperature was controlled to be 25±2° C., the illumination time was 08: 00-20: 00, sufficient feed and drinking water were provided daily, and the padding was replaced twice a week.

2) Test Substance and Route of Administration

The cisplatin (cis, 20 mg/kg) was administered by intraperitoneal injection on the first day. The Antrodia cinnamomea composition (400 mg/kg) was administered in a tube-teed manner on the 1st to 5th day, and the time of tube feeding on the first day was 4 hours after the chemotherapeutic drug was administered. The animal groups were the control group,Cis and Antrodia cinnamomea compositions (AC) respectively, with 6 mice in each group and 18 mice in 3 groups.

3) Assessment of Bone Marrow Hematopoietic Capability

The experimental animals were sacrificed in a CO₂ manner on the sixth day, the thigh bones of the mice were surgically removed, both ends of the bones were cut, bone marrow cells were flushed out, and after centrifugation and washing, the cells were added to MethoCult M3334 medium or MethoCult M3234 medium, and allowed to stand in a humidified incubator containing 5% CO₂ at 37° C. and cultured, the former was cultured for 48 hours to observe by microscope and calculate the number of CFU-E (Colony-forming unit-erythroid) colonies, and the latter was cultured for 7 days to observe by microscope and calculate the number of CFU-GM(Colony-forming unit-granulocyte, macrophage) colonies. The colony numbers of CFU-E and CFU-GM were counted by observation in the whole field of view.

4) Assessment of Peripheral Blood Leukocyte Change

Before the experimental animals were sacrificed, the whole blood was collected in a cheek blood collection manner, and anticoagulant whole blood was used for complete blood count (CBC) to determine the number of peripheral white blood cells.

5) Statistical Analysis Methods

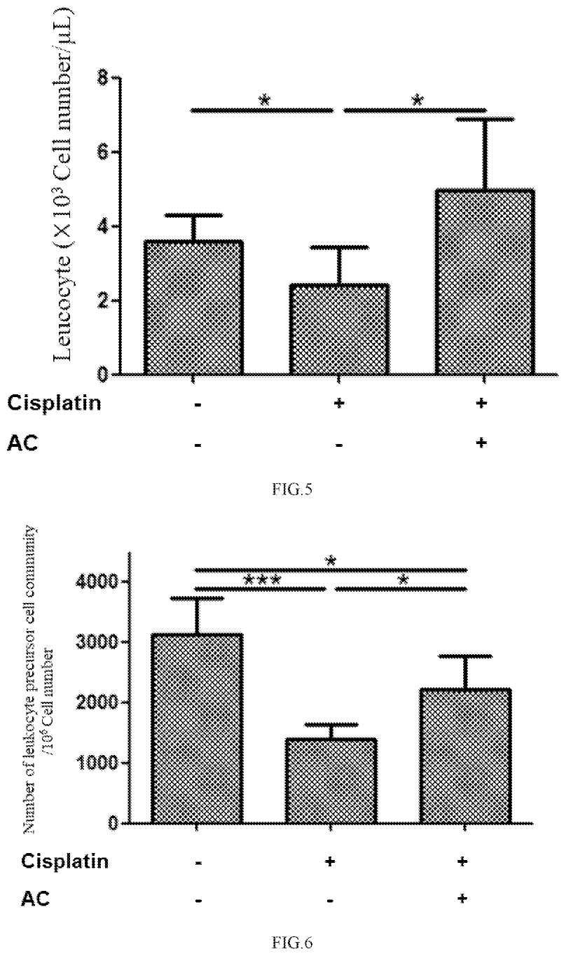

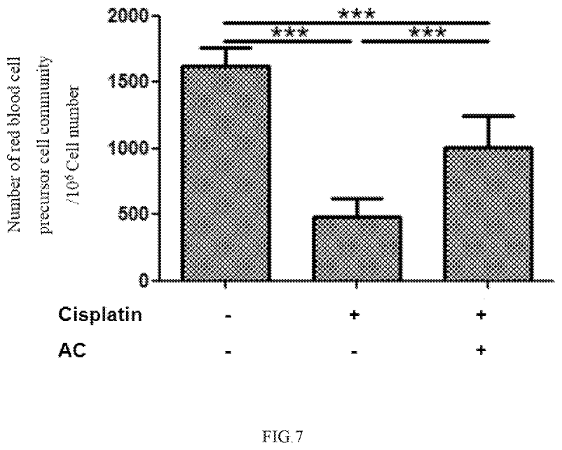

One-way analysis of variance (One-Way ANOVA) was used for data analysis. When the analysis result reached a significant level, Fisher LSD was used for subsequent comparison. The results of animal experiments are shown in FIG. 5, FIG. 6 and FIG. 7.

The results in FIG. 5 shows that the chemotherapy drug cisplatin can reduce the number of white blood cells in immune normal mice. When the Antrodia cinnamomea composition is used in combination with the chemotherapy drug cisplatin, the Antrodia cinnamomea composition can effectively improve the reduction of the number of white blood cells caused by the chemotherapy drug cisplatin, so that it can be restored to be equivalent to the control group. The results in FIG. 6 show that the chemotherapeutic drug cisplatin inhibits the number of myeloid leukocyte precursor cells CFU-GM in immune normal mice. When the Antrodia cinnamomea . composition is used in combination with the chemotherapy drug cisplatin, the Antrodia. cinnamomea composition can effectively improve the bone marrow inhibition caused by the chemotherapy drug cisplatin, so that the number of CFU-GM is significantly improved. The results in FIG. 7 show that the chemotherapeutic drug cisplatin can inhibit the number of bone marrow red blood cell precursor cells CFU-E in immune normal mice. When the Antrodia cinnamomea composition is used in combination with the chemotherapy drug cisplatin, the Antrodia cinnamomea composition can effectively improve bone the marrow inhibition caused by the chemotherapy drug cisplatin, so that the number of CFU-E is significantly improved.

The above results indicate that when the Antrodia cinnamomea composition is used in combination with the chemotherapy drug cisplatin, the side effects of the chemotherapy drug cisplatin on immune normal mice can be improved, including the decrease in the number of white blood cells and the decrease in the number of bone marrow red and white blood cell precursor cells CFU-GM and CFU-E.

In addition, the present invention provides a pharmaceutical composition comprising a crude extract prepared by the above method at a therapeutic effective dose and a pharmaceutically acceptable carrier as appropriate, and is used for preparing medical drugs for preventing or treating colorectal cancer, liver cancer and skin cancer. The medical drug has poison killing effect on colorectal cancer, liver cancer and skin cancer cells.

In addition, the present invention provides a pharmaceutical composition comprising a second sub-extract F2 or a third sub-extract F3 of Antrodia cinnamomea extract prepared by the above method at a therapeutic effective dose, and a pharmaceutically acceptable carrier as appropriate, and is used for preparing medical drugs for preventing or treating colorectal cancer, liver cancer, gastric cancer, lung cancer, breast cancer and skin cancer. The medical drug has poison killing effect on colorectal cancer, liver cancer, gastric cancer, lung cancer, breast cancer and skin cancer cells.

In addition, the present invention also provides a pharmaceutical composition comprising an Antrodia cinnamomea composition prepared by the above method at a. therapeutic effective dose, and a pharmaceutically acceptable carrier as appropriate, and is used for preparing drugs for improving the side effects of the chemotherapeutic drug cisplatin and the platinum-containing series of drugs. The drug is able to improve the number of leukocytes and the phenomenon of bone marrow inhibition caused by cisplatin series drugs of chemotherapy drugs.

Unless otherwise defined herein, the scientific and technical terms used herein should have the meanings understood by those skilled in the art. The meaning and scope of these technical terms should be clear. However, in the case of any potential ambiguity, the definitions provided herein are superior to any dictionary or extrinsic definition. Unless otherwise indicated, the following terms, as used in this disclosure, should be understood to have the following meanings. As used herein, the term “cancer” refers to a disease in which malignant tissue cells grow out of control. Such cell growth may cause transfer, invasion of adjacent tissues. As used herein, the term “treatment” means alleviating or ameliorating symptoms of an illness individual. As used herein, the term “individual” means an animal, in particular a mammal. As used herein, the term “therapeutically effective dose” refers to an amount of active ingredient alone or in combination with other therapeutic/drug combinations to treat cancer display therapeutic efficacy. The term“carrier”or “pharmaceutically acceptable carrier” refers to diluents, excipients, recipients or the like that are well known to those skilled in the art for preparing pharmaceutical compositions.

Those skilled in the art will appreciate that the embodiments of the present invention may readily achieve the goal and obtain the results and advantages mentioned, as well as those present therein. The compositions and methods of preparation of the present invention are representative of preferred embodiments, which are exemplary and are not limited to the field of the invention. Modifications and other uses will occur to those skilled in the art. Such modifications are intended to be included within the spirit of the invention and are within the scope of the invention.

The content description and embodiments of the present invention are disclosed in detail, so that those skilled in the art can make and use the present invention. Even if there are various changes, modifications and advances, they should still be regarded as not departing from the spirit and scope of the present invention.

The invention, as suitably exemplified herein, may be implemented in the absence of any element, or many elements, limitations, or limitations that are not specifically disclosed herein. The terms and expressions used are intended as a description of the specification and are not intended to be limiting, but are not intended to exclude any nouns and expressions equivalent to those shown and described or portions thereof, but it is to be understood that various changes may occur within the scope of the present invention. Therefore, it is to be understood that while the invention has been particularly disclosed in terms of preferred embodiments and any of the features thereof, those skilled in the art will modify and vary the content disclosed therein, and such modifications and variations are still within the scope of the invention.

The preferred embodiment further describes the objects, technical scheme and beneficial effects of the present invention in detail. It should be understood that the foregoing description is only intended to illustrate a specific embodiment of the invention and not to limit the invention. Any modification, equivalent replacement and improvement made to the embodiment without departing from the spirit and principles of the invention should fall within the protection scope of the invention.

1. A method for preparing an Antrodia cinnamomea extract comprising the following steps: using an ethanol solution to extract culture dish-type Antrodia cinnamomea sporocarp powder, concentrating a filtrate under reduced pressure to obtain a crude extract; adsorbing the crude extract by using a macroporous resin and then placing same in a rectangular container; sequentially eluting by using a mixed solution, concentrating an eluate under reduced pressure, and drying to obtain a first sub-extract of an Antrodia cinnamomea extract; next, eluting by using an ethanol solution, concentrating an eluate under reduced pressure, and drying to obtain a second sub-extract of the Antrodia cinnamomea extract; next, eluting by using an ethyl acetate solution, concentrating an eluate under reduced pressure, and drying to obtain a third sub-extract of the Antrodia cinnamomea extract.

2. The method for preparing an Antrodia cinnamomea extract of claim 1, wherein the mixed solution is composed of a secondary water and an ethanol solution; the volume ratio of the secondary water to the ethanol solution is (40-60): (40-60).

3. The method for preparing an Antrodia cinnamomea extract of claim 1, wherein the volume fraction of the ethanol solution is 95%.

4. A method for preparing an Antrodia cinnamomea composition comprises the following steps: extracting .Antrodia cinnamomea mycelium powder by using a secondary water, concentrating a filtrate under reduced pressure to obtain a concentrated solution; adding the concentrated solution into an ethanol solution, filtering to obtain a precipitate; drying the precipitate to obtain an Antrodia cinnamomea mycelium extract; and combining the Antrodia cinnamomea mycelium extract with a second sub-extract of the Antrodia cinnamomea extract prepared by the method of claim 1 to obtain an Antrodia cinnamomea composition.

5. The Antrodia cinnamomea composition of claim 4, wherein the weight ratio of the Antrodia cinnamomea mycelium extract to the second sub-extract of the Antrodia cinnamomea extract is (35-65): (35-65).

6. The Antrodia cinnamomea composition of claim 4, wherein the volume ratio of the concentrated solution to the ethanol solution is 1:3; the volume fraction of the ethanol solution is 60%-80%.

7. A pharmaceutical composition comprising a crude extract prepared by the method of claim 1 at a therapeutic effective dose and a pharmaceutically acceptable carrier as appropriate, and is used for preparing medical drugs for preventing or treating colorectal cancer, liver cancer and skin cancer; the medical drug has poison killing effect on colorectal cancer, liver cancer and skin cancer cells.

8. A pharmaceutical composition comprising a second sub-extract or a third sub-extract of Antrodia cinnamomea extract prepared by the method of claim 1 at a therapeutic effective dose, and a pharmaceutically acceptable carrier as appropriate, and is used for preparing medical drugs for preventing or treating colorectal cancer, liver cancer, gastric cancer, lung cancer, breast cancer and skin cancer; the medical drug has poison killing effect on colorectal cancer, liver cancer, gastric cancer, lung cancer, breast cancer and skin cancer cells.

9. A pharmaceutical composition comprising an Antrodia cinnamomea composition prepared by the method of claim 4 at a therapeutic effective dose, and a pharmaceutically acceptable carrier as appropriate, and is used for preparing drugs for improving the side effects of the chemotherapeutic drug cisplatin and the platinum-containing series of drugs; the drug is able to improve the number of leukocytes and the phenomenon of bone marrow inhibition caused by cisplatin series drugs of chemotherapy drugs.
